(19) United States
(12) Patent Application Publication
(10) Pub. No.: US 2020/0113454 A1
(43) Pub. Date: Apr. 16, 2020

(54) VIBRATION SENSING DEVICE

(71) Applicant: Hong Yue Technology Corporation, Hsinchu City (TW)

(72) Inventors: Chi-Sheng Wu, Hsinchu City (TW); Mei-Hua Liao, Hsinchu City (TW)

(73) Assignee: Hong Yue Technology Corporation, Hsinchu City (TW)

(21) Appl. No.: 16/417,650

(22) Filed: May 21, 2019

(30) Foreign Application Priority Data
Oct. 12, 2018 (TW) 107135985

(51) Int. Cl. A61B 5/021; A61B 5/00; A61B 5/024; A61B 5/0404; A61B 5/0408

(52) U.S. Cl. CPC A61B 5/02108; A61B 5/681; A61B 5/02438; A61B 5/0404; A61B 5/0408; A61B 5/02433

(57) ABSTRACT

A vibration sensing device includes a piezoelectric element, a static force meter, a vibration conduction element and a casing. The piezoelectric element is provided for converting vibration into an electronic signal. The static force meter is provided for converting static force into an electronic signal. The vibration conduction element is provided for vibration to the piezoelectric element. The piezoelectric element, the static force meter, and the vibration conduction element are covered by the casing.

### FIELD OF THE INVENTION

The present invention relates to a physiological signal monitoring device, and more particularly to a portable cardiopulmonary efficacy monitoring device.

### BACKGROUND OF THE INVENTION

Heart disease is the leading cause of death in the United States and the third largest killer in Taiwan. If the heart disease can be diagnosed at an early stage, the survival rate will be increased, and the medical expenditure will be reduced greatly. Most of the early heart diseases have abnormal electrocardiography (ECG) or heart sound symptoms. If a user has easily accessible ECG and heart sound recorders, the user will be able to obtain an ECG and a heart sound immediately to assist doctors' diagnosis when the user feels unwell. Taiwan Pat. Nos. 1555509 and 1623298 (or their foreign counterparts: U.S. Pat. Nos. US20170127966A1 and US20170273574A1 respectively) have disclosed a wearable electrode structure and an ECG recorder for obtaining ECG signals easily. However, there are few recorders capable of recording the heart sound continuously for a long time. The reason is that a sensor (such as a conventional stethoscope) that picks up the heart sound must be pressed slightly at a specific position of a patient's chest. For example, the Holter ECG recorder is uncomfortable to wear for a long time, so that there are few sensors that can record the heart sound continuously for a long time or few heart sound sensors (similar to an ECG event recorder) for obtaining the heart sound when the user feels unwell. As disclosed in U.S. Pat. No. 9,492,138, two electrodes are added to a conventional stethoscope, so that both heart sound and the ECG signal can be measured simultaneously to facilitate the calculation of a myocardial performance index (MPI) and a systolic performance index (SPI), and these indexes may be used for evaluating a patient's heart function. However, the patient must wear clothing to obtain the heart sound and ECG signal. Obviously, the conventional stethoscope is in applicable for recording incidental abnormal heart sounds and ECG signals in our daily life.

Therefore, it is an important subject for the related field to provide a feasible solution to the aforementioned problem.

### SUMMARY OF THE INVENTION

To overcome the aforementioned problem, the present invention provides a vibration sensing device, a portable continuous blood pressure measuring device and a portable cardiopulmonary efficacy monitoring device which can be used conveniently without the need of taking off the patient's clothes.

To achieve the aforementioned objective, the present invention provides a vibration sensing device comprising a piezoelectric element, a static force meter, a vibration conduction element and a casing. The piezoelectric element is provided for converting vibration into an electronic signal. The static force meter is provided for converting static force into an electronic signal. The vibration conduction element is provided for conducting vibration to the piezoelectric element. The casing is provided for covering the piezoelectric element, the static force meter and the vibration conduction element.

The present invention further provides a portable continuous blood pressure measuring device comprising a photo-plethysmograph (PPG)wrist pulse detector, one of the aforementioned vibration sensing devices, a chest pulse filter, a third analog-to-digital converter and a microcontroller. The chest pulse filter is coupled to the vibration sensing device. The third analog-to-digital converter is coupled to the chest pulse filter. The microcontroller is coupled to the photo-plethysmograph (PPG) wrist pulse detector and the third analog-to-digital converter.

The portable continuous blood pressure measuring device further comprises a digital interface coupled to the microcontroller.

The portable continuous blood pressure measuring device further comprises a memory unit coupled to the microcontroller.

The portable continuous blood pressure measuring device further comprises a timing unit coupled to the microcontroller.

In the portable continuous blood pressure measuring device, the digital interface is a cable or wireless signal transmission device.

The present invention further provides a portable continuous blood pressure measuring device comprising the aforementioned vibration sensing device, a heart sound filter, a first analog-to-digital converter, a photo-plethysmograph (PPG) wrist pulse detector and a microcontroller. The heart sound filter is coupled to the vibration sensing device. The first analog-to-digital converter is coupled to the heart sound filter. The microcontroller is coupled to the photo-plethysmograph (PPG) wrist pulse detector and the analog-to-digital converter.

The portable continuous blood pressure measuring device further comprises a digital interface coupled to the microcontroller.

The portable continuous blood pressure measuring device further comprises a memory unit coupled to the microcontroller.

The portable continuous blood pressure measuring device further comprises a timing unit coupled to the microcontroller.

In the portable continuous blood pressure measuring device, the digital interface is a cable or wireless signal transmission device.

The present invention further provides a portable continuous blood pressure measuring device comprising a first electrode, a second electrode, an ECG signal processor, a photo-plethysmograph (PPG) wrist pulse detector, a static force meter and a microcontroller. The first electrode is provided for detecting an ECG signal. The second electrode is configured to be relative to the first electrode. The ECG signal processor is coupled to the first electrode and the second electrode. The static force meter is coupled to the photo-plethysmograph (PPG) wrist pulse detector. The microcontroller is coupled to the photo-plethysmograph (PPG) wrist pulse detector and the ECG signal processor.

The portable continuous blood pressure measuring device further comprises a digital interface coupled to the microcontroller.

The portable continuous blood pressure measuring device further comprises a memory unit coupled to the microcontroller.

The portable continuous blood pressure measuring device further comprises a timing unit coupled to the microcontroller.

In the portable continuous blood pressure measuring device, the digital interface is a cable or wireless signal transmission device.

The present invention further provides a portable continuous blood pressure measuring device comprising a first electrode, a second electrode, an ECG signal processor, the aforementioned vibration sensing device, a heart sound filter, a first analog-to-digital converter and a microcontroller. The first electrode is provided for detecting an ECG signal. The second electrode is configured to be relative to the first electrode. The ECG signal processor is coupled to the first electrode and the second electrode. The heart sound filter is coupled to the vibration sensing device. The first analog-to-digital converter is coupled to the heart sound filter. The microcontroller is coupled to the photo-plethysmograph (PPG) wrist pulse detector and the analog-to-digital converter.

The portable continuous blood pressure measuring device further comprises a digital interface coupled to the microcontroller.

The portable continuous blood pressure measuring device further comprises a memory unit coupled to the microcontroller.

The portable continuous blood pressure measuring device further comprises a timing unit coupled to the microcontroller.

In the portable continuous blood pressure measuring device, the digital interface is a cable or wireless signal transmission device.

The present invention further provides a portable cardiopulmonary efficacy monitoring device comprising a first electrode, a second electrode, an ECG signal processor, the aforementioned vibration sensing device, a heart sound filter, a first analog-to-digital converter, a lung sound filter, a second analog-to-digital converter, a photo-plethysmograph pulse detector and a microcontroller. The first electrode is provided for detecting an ECG signal. The second electrode is configured to be relative to the first electrode. The ECG signal processor is coupled to the first electrode and the second electrode. The heart sound filter is coupled to the vibration sensing device. The second analog-to-digital converter is coupled to the lung sound filter. The microcontroller is coupled to the ECG signal processor, the vibration sensing device, the heart sound filter, the lung sound filter, the red light photo-plethysmograph pulse detector and the infrared photo-plethysmograph pulse detector.

The portable cardiopulmonary efficacy monitoring device further comprises a digital interface coupled to the microcontroller.

The portable cardiopulmonary efficacy monitoring device further comprises a memory unit coupled to the microcontroller.

The portable cardiopulmonary efficacy monitoring device further comprises a timing unit coupled to the microcontroller.

In the portable cardiopulmonary efficacy monitoring device, the digital interface is a cable or wireless signal transmission device.

### BRIEF DESCRIPTION OF THE DRAWINGS

FIG. 1 is a schematic view of a vibration sensing device of the present invention;

FIG. 2 is a schematic block diagram of a portable continuous blood pressure measuring device in accordance with a first embodiment of the present invention;

FIG. 3 is a schematic block diagram of a portable continuous blood pressure measuring device in accordance with a second embodiment of the present invention;

FIG. 4 is a schematic block diagram of a portable continuous blood pressure measuring device in accordance with a third embodiment of the present invention;

FIG. 5 is a schematic block diagram of a portable continuous blood pressure measuring device in accordance with a fourth embodiment of the present invention;

FIG. 6 is a schematic block diagram of a portable cardiopulmonary efficacy monitoring device in accordance with a fifth embodiment of the present invention;

FIG. 7A is a perspective view of a portable cardiopulmonary efficacy monitoring device of the present invention; and

FIG. 7B is a schematic view showing a way of using a wrist watch.

### DESCRIPTION OF THE INVENTION

The technical contents of the present invention will become apparent with the detailed description of preferred embodiments accompanied with the illustration of related drawings as follows. It is intended that the embodiments and figures disclosed herein are to be considered illustrative rather than restrictive.

The present invention is directed to a vibration sensing device operating together with a wristband and a filter to measure a wearer's cardiopulmonary efficacy through clothing without the need of wearing the device for a long time.

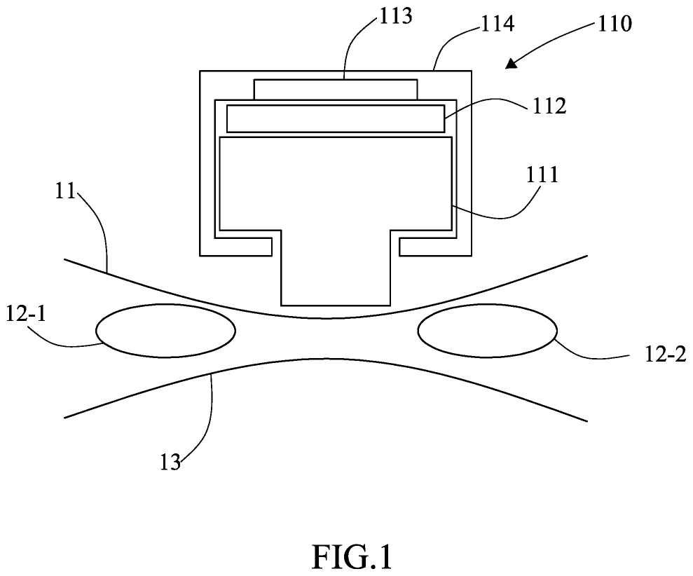

With reference to FIG. 1 for a vibration sensing device of the present invention, the vibration sensing device 110 comprises a piezoelectric element 113, a static force meter 112, a vibration conduction element 111 and a casing 114. The piezoelectric element 113 is a piezo film capable of generating a voltage when the piezo film is pressed, so that the piezoelectric element 113 can convert vibration into an electronic signal. The static force meter 112 is provided for measuring a static force and converting the static force into an electronic signal.

The vibration conduction element 111 is a T-shaped hard conductor provided for conducing vibration and static force to the piezoelectric element 113 and the static force meter 112. Since the piezoelectric element 113 is a film, it may be damaged or its signal may be distorted when the vibration sensing device 110 is applied with a strong force in contact with a user's skin 11 in order to measure the heart sound and lung sound. The vibration conduction element 111 acts as a middle conducting element to conduct the vibration of the heart sound and the lung sound to the piezoelectric element 113, and the static force meter 112 is provided for measuring the applied pressure to prevent over-pressing the piezoelectric element 113.

The static force meter 112 can compensate the deficiency of the piezoelectric element 113 that cannot measure the static force (in form of a DC signal). On the other hand, the piezoelectric element 113 can also compensate the deficiency of the static force meter 112 that cannot measure the vibration (in form of an AC signal). Therefore, the static force meter 112 and the piezoelectric element 113 allow the vibration sensing device 110 to measure both the signals of the static force and the vibration. The casing 114 is provided for covering the static force meter 112, the piezoelectric element 113 and a part of the vibration conduction element 111. The other part of the vibration conduction element 111 is protruded from the casing 114 and contacted with the user's skin 11. In an embodiment as shown in FIG. 1, the static force meter 112 is installed between the piezoelectric element 113 and the vibration conduction element 111. In a preferred embodiment, the positional relation between the static force meter 112 and the piezoelectric element 113 is interchangeable, so that the position of the static force meter 112 and the position of the piezoelectric element 113 may be switched to play the original effect.

In a specific way of using the vibration sensing device 110, an end point of the vibration conduction element 111 protruded out from the casing 114 is in contact with the user's skin 11, so that the vibration sensing device 110 can receive the vibration of the heart or lung from a position between the two ribs 12-1, 12-2 and further measure the heart sound or lung sound. The electronic signal obtained from a vibration measured by the vibration sensing device 110 can be calculated as different data by an external device in order to monitor the cardiopulmonary efficacy, and applications of different embodiments will be described below.

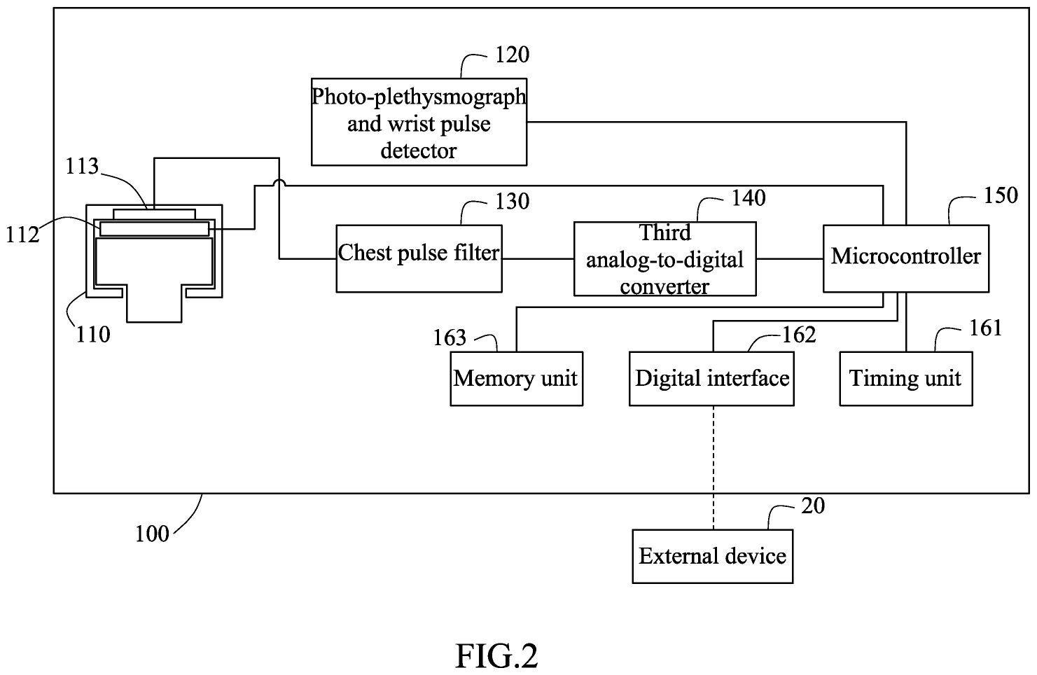

With reference to FIG. 2 for a portable continuous blood pressure measuring device in accordance with a first embodiment of the present invention, the portable continuous blood pressure measuring device 100 comprises the aforementioned vibration sensing device 110 (including the piezoelectric element 113 and the static force meter 112), a photo-plethysmograph (PPG) wrist pulse detector 120, a chest pulse filter 130, a third analog-to-digital converter 140 and a microcontroller 150. In a preferred embodiment, the portable continuous blood pressure measuring device 100 further comprises a digital interface 162, a memory unit 163 and a timing unit 161 which will be described in detail below.

The photo-plethysmograph (PPG) wrist pulse detector 120 is coupled to the microcontroller 150, and the photo-plethysmograph (PPG) wrist pulse detector 120 obtains a wrist pulse by a photo-plethysmograph method and transmits the wrist pulse to the microcontroller 150.

The chest pulse filter 130 is coupled to the piezoelectric element 113 of the vibration sensing device 110. In an embodiment, the chest pulse filter 130 can filter the signal measured by the piezoelectric element 113 to generate the chest pulse. The third analog-to-digital converter 140 is coupled to the chest pulse filter 130 and provided for receiving an electronic signal received by the chest pulse filter 130. Further, the electronic signal is converted into a digital signal. The microcontroller 150 is provided for receiving the signals from the third analog-to-digital converter 140 and the photo-plethysmograph (PPG) wrist pulse detector 120. In this embodiment, the microcontroller 150 obtains the chest pulse and the wrist pulse and then calculates the time between the chest pulse and the wrist pulse which is a pulse transit time (PTT), and a continuous blood pressure can be calculated by the PTT, so as to measure a continuous blood pressure.

The digital interface 162 is coupled to the microcontroller 150, and the digital interface 162 is a cable or wireless signal transmission device provided for transmitting the information received by the digital interface 162 to an external device 20. The memory unit 163 is coupled to the microcontroller 150 and provided for storing the information computed by the microcontroller 150. The timing unit 161 is coupled to the microcontroller 150 for providing time information to the microcontroller 150. The external device 20 includes but not limited to a smartphone or a personal computer, and the external device 20 is installed with software and coupled to the portable continuous blood pressure measuring device 100. The external device 20 receives and then displays related data for the view of others.

In the first embodiment, the static force meter 112 is coupled to the microcontroller 150, and the external device 20 to remind the user to apply an appropriate static force to obtain good-quality chest pulses and wrist pulses and prevent damaging the piezoelectric element.

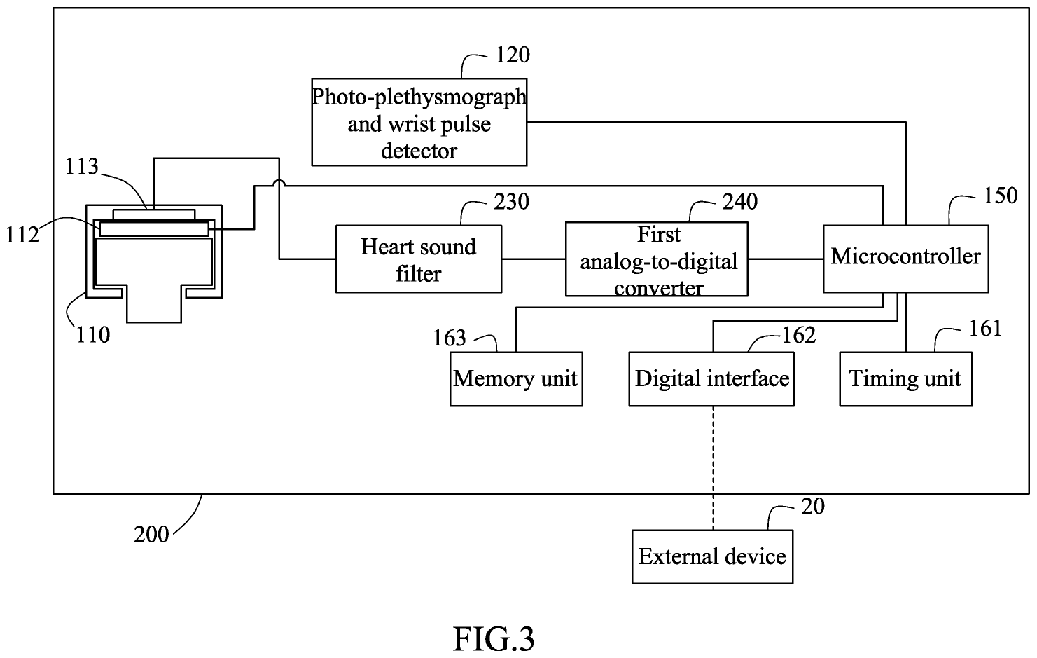

With reference to FIG. 3 for a portable continuous blood pressure measuring device in accordance with a second embodiment of the present invention, the portable continuous blood pressure measuring device 200 of this embodiment comprises the aforementioned vibration sensing device 110 (including the piezoelectric element 113 and the static force meter 112), a photo-plethysmograph (PPG) wrist pulse detector 120, a heart sound filter 230, a first analog-to-digital converter 240 and a microcontroller 150. In a preferred embodiment, the portable continuous blood pressure measuring device 200 further comprises a digital interface 162, a memory unit 163 and a timing unit 161. Wherein, the functions of the vibration sensing device 110, the photo-plethysmograph (PPG) wrist pulse detector 120, the digital interface 162, the memory unit 163 and the timing unit 161 are the same of the previous embodiment, and thus will not be repeated here.

The second embodiment is characterized in that the portable continuous blood pressure measuring device 200 further comprises a heart sound filter 230 and a first analog-to-digital converter 240. The heart sound filter 230 is coupled to the piezoelectric element 113 of the vibration sensing device 110 and capable of filtering the electronic signal of the piezoelectric element 113 to generate a heart sound, and the first analog-to-digital converter 240 is coupled to the heart sound filter 230 and provided for converting the heart sound into a digital signal. The microcontroller 150 receives the heart sound and the wrist pulse and then computes the time between the heart sound and the wrist pulse which is the pulse transit time (PTT), and a continuous blood pressure can be calculated by the PTT, so as to measure the continuous blood pressure.

In the second embodiment, the portable continuous blood pressure measuring device 200 further comprises a static force meter 112 coupled to the microcontroller 150, and an external device 20 is provided for reminding a user to apply an appropriate static force to obtain good-quality heart sounds and wrist pulses and prevent damaging the piezoelectric element.

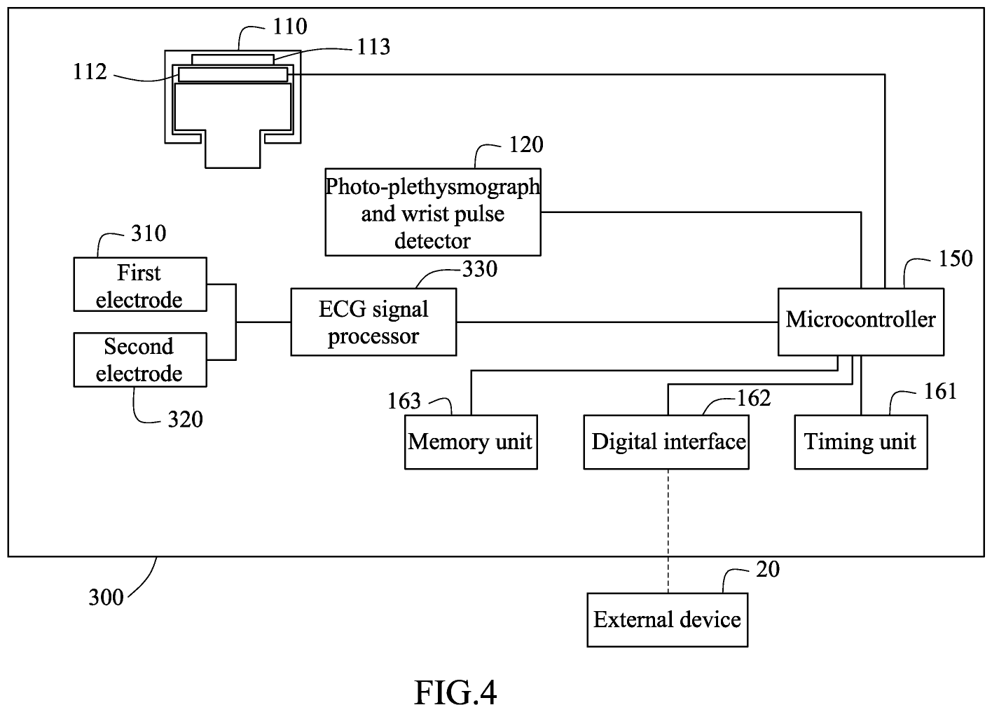

With reference to FIG. 4 for a portable continuous blood pressure measuring device in accordance with a third embodiment of the present invention, the portable continuous blood pressure measuring device 300 of this embodiment comprises a first electrode 310, a second electrode 320, an ECG signal processor 330, a static force meter 112, a photo-plethysmograph (PPG) wrist pulse detector 120, a microcontroller 150, a digital interface 162, a memory unit 163 and a timing unit 161. Wherein, the functions of the photo-plethysmograph (PPG) wrist pulse detector 120, the digital interface 162, the memory unit 163, and the timing unit 161 are the same as those of the previous embodiment, and thus will not be repeated here.

The third embodiment is characterized in that the portable continuous blood pressure measuring device 300 comprises a first electrode 310, a second electrode 320, an ECG signal processor 330 and a static force meter 112. The first electrode 310 and the second electrode 320 are provided by measuring an ECG signal by contacting the first electrode 310 and the second electrode 320 with a wearer's skin. The ECG signal processor 330 is coupled to the first electrode 310 and the second electrode 320 for processing the ECG signal, and converting the ECG signal into a signal computable by the microcontroller 150. The photo-plethysmograph (PPG) wrist pulse detector 120 is provided for measuring a wrist pulse. The microcontroller 150 is provided for receiving the ECG signal and the wrist pulse and computing the time of the ECG signal and the wrist pulse which is a pulse transit time (PTT), and a continuous blood pressure can be calculated by the PTT, so as to achieve the effect of measuring the continuous blood pressure.

The static force meter 112 is provided for measuring the force applied to the wrist vessel when the photo-plethysmograph (PPG) wrist pulse detector 120 detects a wrist pulse and transmitting the measured value of the force to the microcontroller 150. The microcontroller 150 will recognize the measured value of the force and then display the value of the force on a digital interface 162. If the value of the force is too large or too small, then a warning message will be displayed on the digital interface to remind the user to control the force applied, so as to improve the accuracy of measuring the continuous blood pressure.

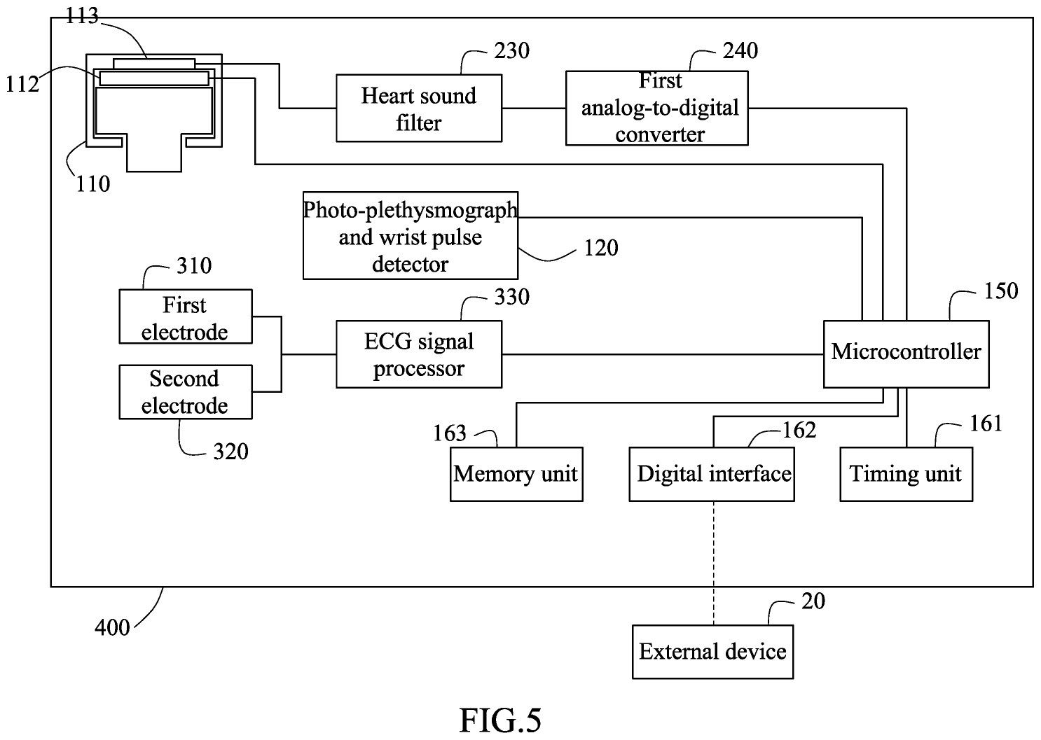

With reference to FIG. 5 for a portable continuous blood pressure measuring device in accordance with a fourth embodiment of the present invention, the portable continuous blood pressure measuring device 400 of this embodiment comprises the aforementioned vibration sensing device 110, a photo-plethysmograph (PPG) wrist pulse detector 120, a heart sound filter 230, a first analog-to-digital converter 240, a first electrode 310, a second electrode 320, an ECG signal processor 330, a static force meter 112, a photo-plethysmograph (PPG) wrist pulse detector 120, a microcontroller 150, a digital interface 162, a memory unit 163 and a timing unit 161.

Specifically, the fourth embodiment combines the advantages of the third embodiment and the second embodiment. The first electrode 310 and second electrode 320 are provided for measuring an ECG signal, and the vibration sensing device 110 is provided measuring a heart sound signal, and the photo-plethysmograph (PPG) wrist pulse detector 120 is provided for measuring a wrist pulse and operating with a heart sound filter 230 and an ECG signal processor 330 for processing and providing appropriate ECG signals and heart sound signals. The microcontroller 150 receives the wrist pulse, ECG signal and heart sound signal and further calculates the pulse transmit time and estimates a continuous blood pressure to achieve the effect of measuring the continuous blood pressure. In the meantime, the vibration sensing device 110 further comprises a static force meter 112 capable of applying a force to the wrist vessel when the photo-plethysmograph (PPG) wrist pulse detector 120 detects a wrist pulse and uses the applied force as a reference for the user to ensure the accuracy of measuring the continuous blood pressure.

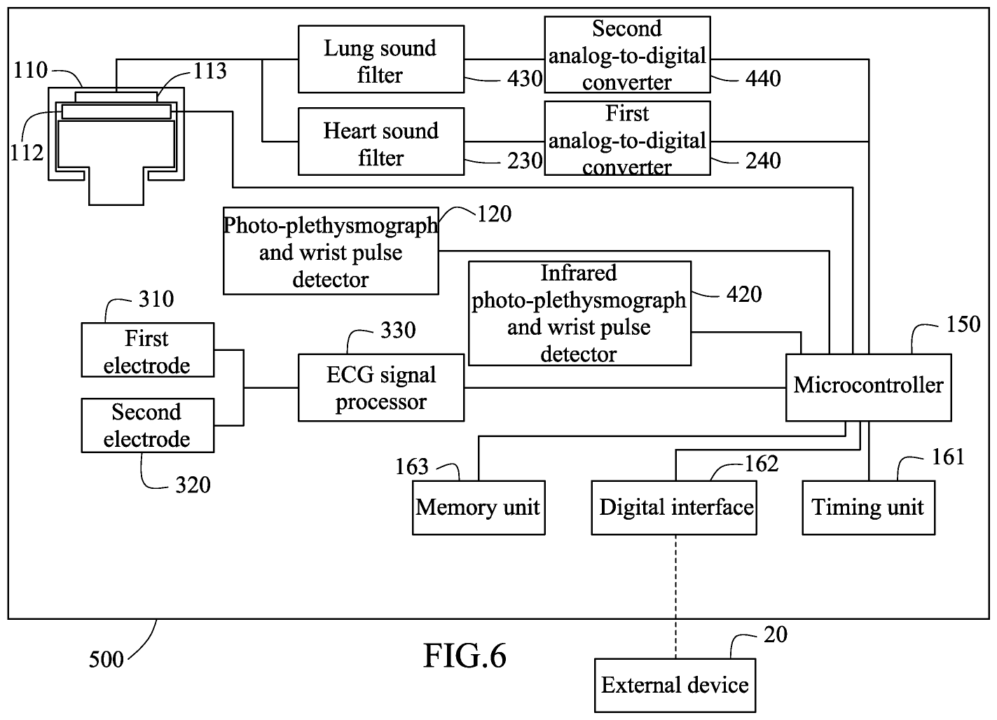

With reference to FIG. 6 for a portable continuous blood pressure measuring device in accordance with a fifth embodiment of the present invention, the portable cardiopulmonary efficacy monitoring device 500 of this embodiment comprises the aforementioned vibration sensing device 110 (including a piezoelectric element 113 and a static force meter 112), a lung sound filter 430, a second analog-to-digital converter 440, an infrared photo-plethysmograph (PPG) wrist pulse detector 420, a photo-plethysmograph (PPG) wrist pulse detector 120, a heart sound filter 230, a first analog-to-digital converter 240, a first electrode 310, a second electrode 320, an ECG signal processor 330, a photo-plethysmograph (PPG) wrist pulse detector 120, a microcontroller 150, a digital interface 162, a memory unit 163, and a timing unit 161.

Wherein, the functions of the vibration sensing device 110, the photo-plethysmograph (PPG) wrist pulse detector 120, the heart sound filter 230, the first analog-to-digital converter 240, the first electrode 310, the second electrode 320, the ECG signal processor 330, the microcontroller 150, the digital interface 162, the memory unit 163 and the timing unit 16 are the same as those of the previous embodiments, and thus will not be repeated here.

The portable cardiopulmonary efficacy monitoring device 500 further comprises a lung sound filter 430, a second analog-to-digital converter 440 and an infrared photo-plethysmograph (PPG) wrist pulse detector 420. The lung sound filter 430 is coupled to the vibration sensing device 110 and provided for receiving an electronic signal from the vibration sensing device 110. The lung sound filter 430 filters an electronic signal transmitted from the vibration sensing device 110 to generate a lung sound signal. The second analog-to-digital converter 440 is coupled to the lung sound filter 430 and provided for converting the analog lung sound signal filtered by the lung sound filter 430 into a digital signal and then transmitting the digital signal to the microcontroller 150. The infrared photo-plethysmograph (PPG) wrist pulse detector 420 detects a user's wrist pulse by infrared light and transmits the wrist pulse to the controller 150.

In the fifth embodiment, the portable cardiopulmonary efficacy monitoring device 500 measures the wrist pulse, heart sound, lung sound and ECG signal. The microcontroller 150 integrates and receives these signals and compares the signals with each other to compute parameters such as a myocardial performance index (MPI) and a systolic performance index (SPI) to further analyze the user's human health status.

In the fifth embodiment, the static force meter 112 is coupled to the microcontroller 150, and an external device 20 is provided for reminding a user to apply a static force appropriately to obtain good-quality heart sounds and lung sounds and prevent damaging the piezoelectric element 113.

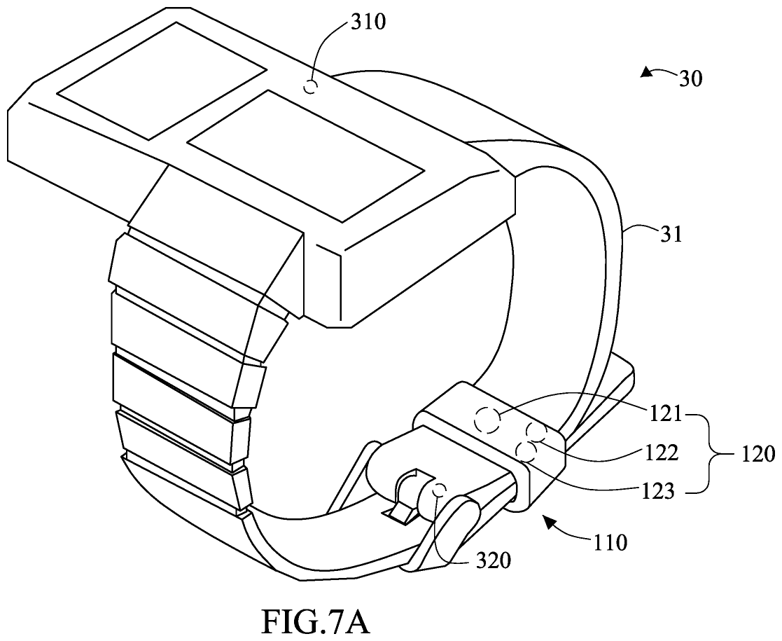

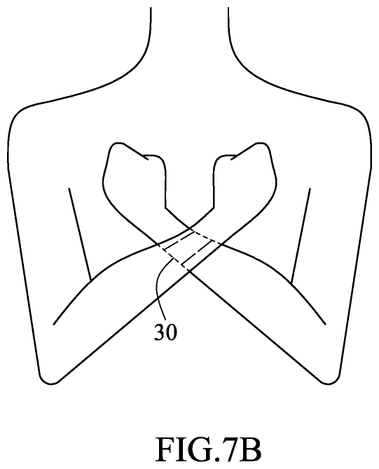

With reference to FIG. 7A for a schematic view of a portable cardiopulmonary efficacy monitoring device in accordance with an embodiment of the present invention, the portable continuous blood pressure measuring device may be in form of a wrist watch 30 provided to be worn by users. The wrist watch 30 may be installed with a portable continuous blood pressure measuring device and contacted the wearer with a photo-plethysmograph wrist pulse detector 120, the vibration sensing device 110, the first electrode 310, and the second electrode 320. In this embodiment, the photo-plethysmograph wrist pulse detector 120 comprises an infrared light source 121, a red light source 122 and a photoreceptor 123 installed on a watchband 31, and the vibration sensing device 110 is installed on the other side of the photo-plethysmograph wrist pulse detector 120. The first electrode 310 and second electrode 320 are installed at relative positions of the watchband 31 respectively. In addition, the first electrode 310 is disposed on an outer side of the wrist watch 30, and the second electrode 320 is disposed on an inner side of the wrist watch 30, so that the first electrode 310 and second electrode 320 can touch a wearer's hands to measure and obtain a signal.

With reference to FIG. 7B for a way of using a wrist watch, a user wears the wrist watch 30 on one of the hands, and then places the wrist watch on his/her chest wall near the heart and covers the wrist watch 30 by the other hand. The user applies a pressure appropriately to keep the wrist watch in touch of the user's body. During use, it is not necessary to take off clothing. Such operation allows the wrist watch to obtain signals such as the wear's ECG, lung sound, heart sound, wrist pulse and chest pulse. The digital interface 162 is provided for reading the signal and transmitting the signal to an external device 30 for display.

The portable continuous blood pressure measuring device or portable cardiopulmonary efficacy monitoring device of the present invention comes with the vibration sensing device 110 having the static force meter 112 and operates with the vibration conduction element 111 to conduct vibration, so that the piezoelectric element 113 can read the numerical values more accurately. These devices may be designed in form of the wrist watch 30 to provide an easy operation and allows users to measure physiological signals through clothing. In addition, the digital interface 162 is provided for transmitting data to other external devices in order to control the physiological signals more easily.

While the present invention has been described by means of specific embodiments, numerous modifications and variations could be made thereto by those skilled in the art without departing from the scope and spirit of the present invention set forth in the claims.

What is claimed is:

1. A vibration sensing device, comprising:
a piezoelectric element, for converting vibration into an electronic signal;
a static force meter, for converting static force into an electronic signal;
a vibration conduction element, for conducting vibration to the piezoelectric element; and
a casing, for covering the piezoelectric element, the static force meter and the vibration conduction element.

2. A portable continuous blood pressure measuring device, comprising:
a photo-plethysmograph (PPG) wrist pulse detector;
a vibration sensing device according to claim 1;
a chest pulse filter, coupled to the vibration sensing device;
a third analog-to-digital converter, coupled to the chest pulse filter; and
a microcontroller, coupled to the photo-plethysmograph (PPG) wrist pulse detector and the third analog-to-digital converter.

3. The portable continuous blood pressure measuring device of claim 2, further comprising a digital interface coupled to the microcontroller.

4. The portable continuous blood pressure measuring device of claim 2, further comprising a timing unit coupled to the microcontroller.

5. The portable continuous blood pressure measuring device of claim 3, wherein the digital interface is a cable or wireless signal transmission device.

6. A portable continuous blood pressure measuring device, comprising:
a vibration sensing device according to claim 1;
a heart sound filter, coupled to the vibration sensing device;
a first analog-to-digital converter, coupled to the heart sound filter;
a photo-plethysmograph (PPG) wrist pulse detector; and
a microcontroller, coupled to the photo-plethysmograph (PPG) wrist pulse detector and the analog-to-digital converter.

7. The portable continuous blood pressure measuring device of claim 6, further comprising a digital interface coupled to the microcontroller.

8. The portable continuous blood pressure measuring device of claim 6, further comprising a timing unit coupled to the microcontroller.

9. The portable continuous blood pressure measuring device of claim 6, wherein the digital interface is a cable or wireless signal transmission device.

10. A portable continuous blood pressure measuring device, comprising:
a first electrode, for detecting an ECG signal;
a second electrode, configured relative to the first electrode;
an ECG signal processor, coupled to the first electrode and the second electrode;
a photo-plethysmograph (PPG) wrist pulse detector;
a static force meter, coupled to the photo-plethysmograph (PPG) wrist pulse detector; and
a microcontroller, coupled to the optical photo-plethysmograph (PPG) wrist pulse detector, and the ECG signal processor.

11. The portable continuous blood pressure measuring device of claim 10, further comprising a digital interface coupled to the microcontroller.

12. The portable continuous blood pressure measuring device of claim 10, further comprising a timing unit coupled to the microcontroller.

13. The portable continuous blood pressure measuring device of claim 11, wherein the digital interface is a cable or wireless signal transmission device.

14. A portable continuous blood pressure measuring device, comprising:
a first electrode, for detecting an ECG signal;
a second electrode, configured to be relative to the first electrode;
an ECG signal processor, coupled to the first electrode and the second electrode;
a vibration sensing device according to claim 1;
a heart sound filter, coupled to the vibration sensing device;
a first analog-to-digital converter, coupled to the heart sound filter; and
a microcontroller, coupled to the photo-plethysmograph (PPG) wrist pulse detector and the analog-to-digital converter.

15. The portable continuous blood pressure measuring device of claim 14, further comprising a digital interface coupled to the microcontroller.

16. The portable continuous blood pressure measuring device of claim 14, further comprising a timing unit coupled to the microcontroller.

17. The portable continuous blood pressure measuring device of claim 15, wherein the digital interface is a cable or wireless signal transmission device.

18. A portable cardiopulmonary efficacy monitoring device, comprising:
a first electrode, for detecting an ECG signal;
a second electrode, configured to be relative to the first electrode;
an ECG signal processor, coupled to the first electrode and the second electrode;
a vibration sensing device according to claim 1;
a heart sound filter, coupled to the vibration sensing device;
a first analog-to-digital converter, coupled to the heart sound filter;
a lung sound filter, coupled to the vibration sensing device;
a second analog-to-digital converter, coupled to the lung sound filter;
a photo-plethysmograph pulse detector;
an infrared photo-plethysmograph pulse detector; and
a microcontroller, coupled to the ECG signal processor, the vibration sensing device, the heart sound filter, the lung sound filter, the photo-plethysmograph pulse detector and the infrared photo-plethysmograph pulse detector.

19. The portable cardiopulmonary efficacy monitoring device of claim 18, further comprising a digital interface coupled to the microcontroller.

20. The portable cardiopulmonary efficacy monitoring device of claim 18, further comprising a timing unit coupled to the microcontroller.

21. The portable cardiopulmonary efficacy monitoring device of claim 19, wherein the digital interface is a cable or wireless signal transmission device.
